(19) United States
(12) Patent Application Publication
(10) Pub. No.: US 2016/0262786 A1
(43) Pub. Date: Sep. 15, 2016

(54) SURGICAL BLADES WITH FATIGUE RESISTANT PROPERTIES

(71) Applicant: Ethicon Endo-Surgery, LLC, Guaynabo, PR (US)

(72) Inventors: Ashvani K. Madan, Mason, OH (US); William A. Olson, Lebanon, OH (US); David A. Witt, Maineville, OH (US)

(21) Appl. No.: 15/062,577

(22) Filed: Mar. 7, 2016

Related U.S. Application Data
(60) Provisional application No. 62/130,892, filed on Mar. 10, 2015.

(51) Int. Cl. A61B 17/32; C21D 7/06; B21D 31/06

(52) U.S. Cl. CPC A61B 17/320068; B21D 31/06; C21D 7/06; A61B 2017/32116

(57) ABSTRACT

A method is disclosed for treating an ultrasonic end effector or surgical blade. The method includes providing an ultrasonic end effector or surgical blade and inducing a residual compressive stress along at least one surface of the ultrasonic end effector or surgical blade. The residual compressive stress may be induced by peening the at least one surface. Such treatment improves the resistance of the ultrasonic end effector or surgical blade to fracture propagation and improves the fatigue life of the ultrasonic end effector or surgical blade. Ultrasonic surgical instruments comprising an ultrasonic end effector or surgical blade treated according to the method are also disclosed.

### CROSS-REFERENCE TO RELATED APPLICATIONS

This application claims the benefit under 35 U.S.C. §119(e) of U.S. Provisional Patent Application Ser. No. 62/130,892, entitled SURGICAL BLADES WITH FATIGUE RESISTANT PROPERTIES, filed Mar. 10, 2015, the entire disclosure of which is hereby incorporated by reference herein.

### BACKGROUND

The present disclosure is related generally to surgical instruments including ultrasonic instruments. Ultrasonic surgical instruments, such as ultrasonic scalpels, are used in many applications in surgical procedures by virtue of their unique performance characteristics. Ultrasonic surgical instruments can be configured for open surgical use, laparoscopic, or endoscopic surgical procedures including robotic-assisted procedures.

### DRAWINGS

The features of the various embodiments are set forth with particularity in the appended claims. The various embodiments, however, both as to organization and methods of operation, together with advantages thereof, may best be understood by reference to the following description, taken in conjunction with the accompanying drawings as follows:

FIG. 1 illustrates an exploded perspective view of an ultrasonic surgical instrument according to one embodiment;

FIG. 2 illustrates a side view of the distal end of an ultrasonic transmission assembly according to one embodiment;

FIG. 2A illustrates a top view of the distal end of an ultrasonic transmission assembly according to one embodiment;

FIG. 3 illustrates a perspective view of the distal end of an ultrasonic transmission assembly according to one embodiment;

FIG. 3A illustrates a perspective view of the distal end of an ultrasonic transmission assembly according to one embodiment;

FIG. 3B illustrates a perspective view of the distal end of an ultrasonic transmission assembly according to one embodiment;

FIG. 3C illustrates a perspective view of the distal end of an ultrasonic transmission assembly according to one embodiment;

FIG. 4 illustrates a perspective view of an untreated ultrasonic blade;

FIG. 5 illustrates a perspective view of a partially treated ultrasonic blade in accordance with various treatment methods described herein;

FIG. 5A illustrates a stress contour plot of an ultrasonic surgical instrument according to one embodiment;

FIG. 5B illustrates a close-up of the ultrasonic surgical instrument of FIG. 5A according to one embodiment;

FIG. 5C illustrates a cross-sectional view of an ultrasonic end effector comprising a peened supporting portion according to one embodiment;

FIG. 6 illustrates a life stress plot of several treated and untreated ultrasonic blades in accordance with various embodiments described herein; and

FIG. 7 illustrates a plot illustrating a relation between a depth of residual stress in a treated blade and corresponding ultrasonic stress magnitude according to one embodiment.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and illustrative. Variations and changes thereto may be made without departing from the scope of the claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation. Furthermore, it will be appreciated that for conciseness and clarity, spatial terms such as “vertical,” “horizontal,” “up,” and “down”, for example, may be used herein with respect to the illustrated embodiments. However, these terms are used to assist the reader and are not intended to be limiting and absolute.

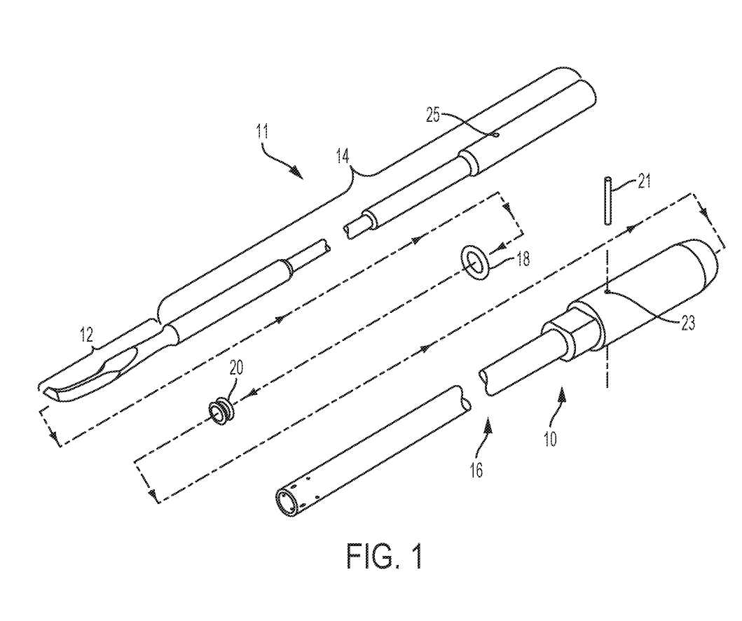

FIG. 1 is an exploded perspective view of an ultrasonic surgical instrument 10 according to the present disclosure. In FIG. 1, ultrasonic end effector 12 is mechanically coupled to ultrasonic transmission waveguide 14 to form ultrasonic transmission assembly 11. Ultrasonic transmission waveguide 14 is positioned in outer sheath 16 by mounting o-ring 18 and sealing ring 20. One or more additional dampers or support members (not shown) may also be included along ultrasonic transmission waveguide 14. Ultrasonic transmission waveguide 14 is affixed to outer sheath 16 by mounting pin 21, which passes through mounting holes 23 in outer sheath 16 and mounting slot 25 in ultrasonic transmission waveguide 14.

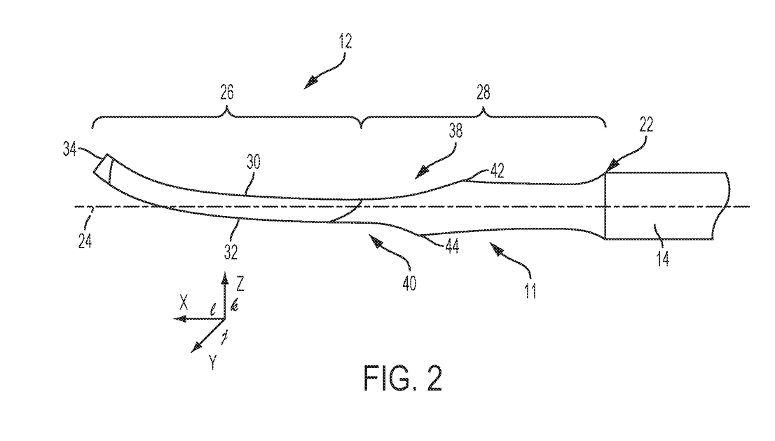

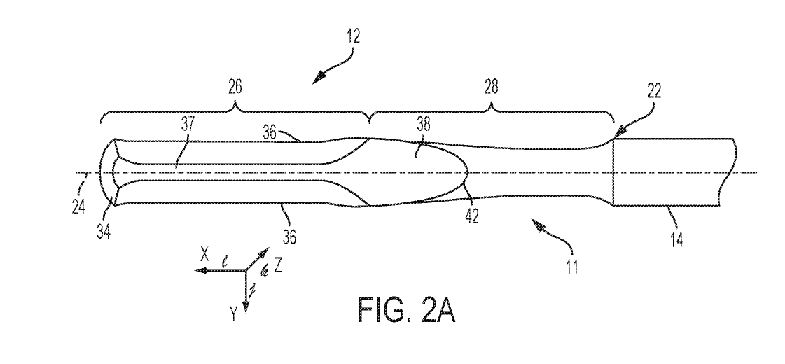

FIG. 2 is a side view of the distal end of ultrasonic transmission assembly 11, including end effector 12. FIG. 2 further includes a coordinate system in which: the x-axis lies along central axis 24 of transmission waveguide 14 while the y-axis is the axis of curvature of treatment region 26 and the z-axis is perpendicular to the x-axis and y-axis. In the embodiments of the invention described herein, end effector 12 is affixed to the distal end of transmission waveguide 14 at balance node 22. Central axis 24 of transmission waveguide 14 extends from the proximal end of transmission waveguide 14 to the distal end of transmission waveguide 14. Transmission waveguide 14 can be symmetrical about central axis 24. End effector 12 includes a working portion or a treatment region 26, which is located at the distal end of end effector 12. A support portion or balance region 28 is located proximal to the treatment region 26. In certain instances, the balance region 28 is located between the treatment region 26 and a balance node 22, as illustrated in FIG. 2. The treatment region 26 can be curved and may include two surfaces, a concave top surface 30 and a convex bottom surface 32, for example. Convex bottom surface 32 can be substantially planar or flat along the y-axis of the treatment region 26. Treatment region 26 may further include a rounded tip 34. In the illustrated embodiment, the balance region 28 includes a first cutout 38 and a second cutout 40 which act as asymmetric balance features. First cutout 38 extending from a proximal end of concave surface 30 to a first predetermined point 42 which is distal to balance node 22. Second cutout 40 extends from a proximal end of convex surface 32 to a second predetermined point 44 which is distal to point 42 and balance node 22.

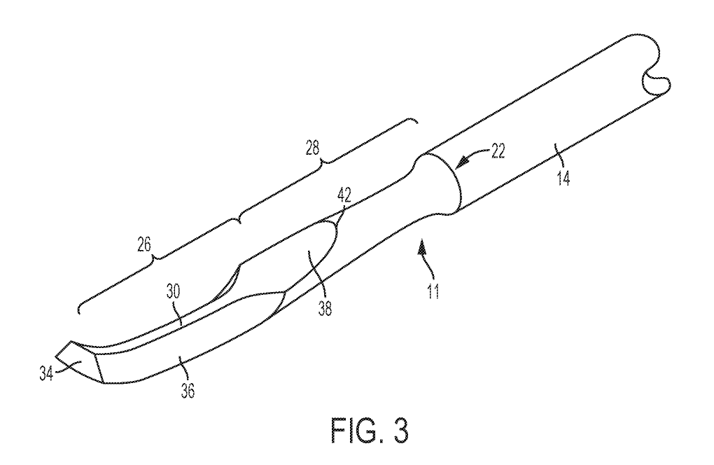

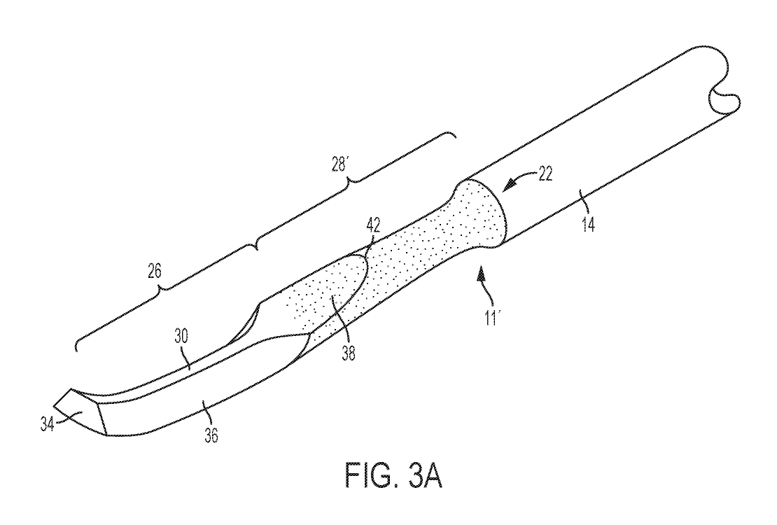

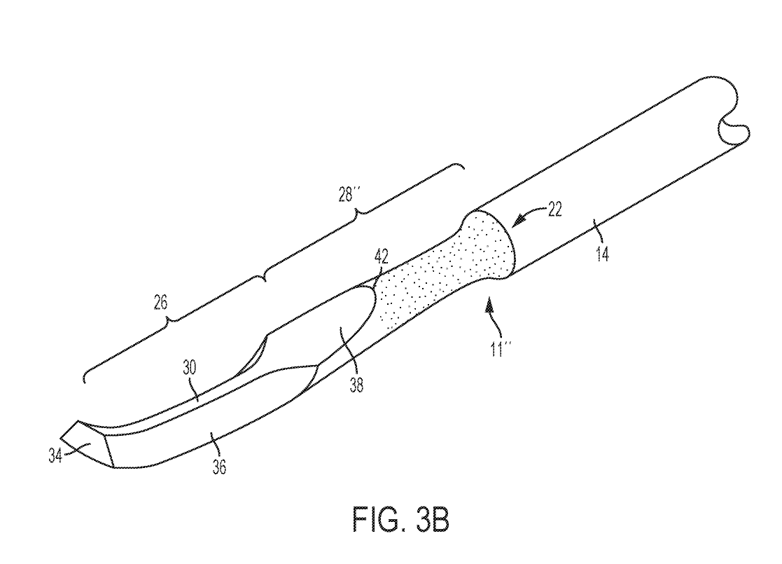

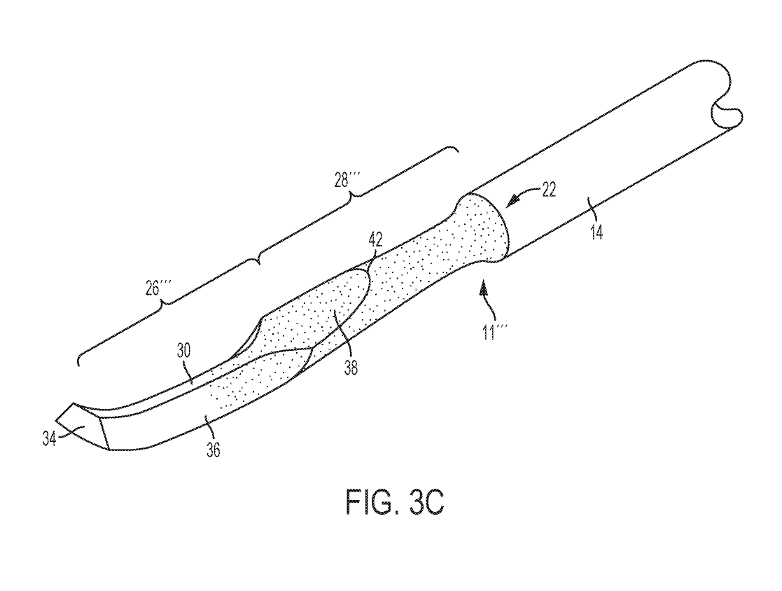

Referring to FIGS. 2A and 3, an embodiment of the distal end of ultrasonic transmission assembly 11 is illustrated. In certain instances, blade edges 36 are positioned on both sides of treatment region 26 and extend from the proximal end of treatment region 26 to rounded tip 34. Central ridge 37 may run from the distal end of balance region 28 to the rounded tip 34 along the center of treatment region 26. Various other embodiments of the end effector 12 may comprise treatment regions/working portions 26 and balance regions/supporting portions 28 with various other geometries.

In various instances, a generator (not shown) may be activated to apply ultrasonic energy to the end effector 12. When the generator is activated, electrical energy is applied by the generator to a transducer stack or assembly, which may cause the treatment region 26 to vibrate at ultrasonic frequencies in the range of, for example, approximately 20 kHz to 250 kHz. The generator can comprise any suitable generator, such as Model No. GEN04, and/or Model No. GEN11 available from Ethicon Endo-Surgery, Inc.

A suitable ultrasonic generator may be configured to functionally operate in a manner similar to the GEN300 sold by Ethicon Endo-Surgery, Inc. of Cincinnati, Ohio as is disclosed in one or more of the following U.S. patents, all of which are incorporated by reference herein in their entireties: U.S. Pat. No. 6,480,796, entitled METHOD FOR IMPROVING THE START UP OF AN ULTRASONIC SYSTEM UNDER ZERO LOAD CONDITIONS; U.S. Pat. No. 6,537,291, entitled METHOD FOR DETECTING A LOOSE BLADE IN A HAND PIECE CONNECTED TO AN ULTRASONIC SURGICAL SYSTEM; U.S. Pat. No. 6,662,127, entitled METHOD FOR DETECTING PRESENCE OF A BLADE IN AN ULTRASONIC SYSTEM; U.S. Pat. No. 6,977,495, entitled DETECTION CIRCUITRY FOR SURGICAL HANDPIECE SYSTEM; U.S. Pat. No. 7,077,853, entitled METHOD FOR CALCULATING TRANSDUCER CAPACITANCE TO DETERMINE TRANSDUCER TEMPERATURE; U.S. Pat. No. 7,179,271, entitled METHOD FOR DRIVING AN ULTRASONIC SYSTEM TO IMPROVE ACQUISITION OF BLADE RESONANCE FREQUENCY AT STARTUP; and U.S. Pat. No. 7,273,483, entitled APPARATUS AND METHOD FOR ALERTING GENERATOR FUNCTION IN AN ULTRASONIC SURGICAL SYSTEM. Furthermore, U.S. Patent Application Publication No. 2014/0005702, entitled ULTRASONIC SURGICAL INSTRUMENTS WITH DISTALLY POSITIONED TRANSDUCERS, filed on Jun. 29, 2012, is incorporated by reference herein in its entirety.

In various instances, a clamp member (not shown) may be movably coupled to the end effector 12. The clamp member can be moved relative to the end effector 12 to capture tissue therebetween. The captured tissue can then be treated by applying ultrasonic energy to the end effector 12, as described above.

As described above, ultrasonic instruments are often used in surgery to cut and coagulate tissue. Exciting an ultrasonic end effector or blade such as, for example, the end effector 12 at ultrasonic frequencies induces vibratory movement which generates localized heat within adjacent tissue facilitating both cutting and coagulation. The structural stress induced in such an end effector or blade by vibrating at ultrasonic frequencies may lead to the formation of cracks or fractures in the body of the end effector or blade. This undesirable effect may be compounded when an excited end effector or blade comes in contact with a hard object such as an end effector of another surgical instrument. Incidental contact between an end effector or blade vibrating at ultrasonic frequencies and a hard object may augment the likelihood of the formation of fractures or cracks in the body of the end effector or blade. Such fractures or cracks tend to propagate through the body of the end effector or blade ultimately leading to failure of the end effector or blade. The speed with which such cracks or fractures propagate through the body of the end effector or blade may influence the fatigue life of the end effector or blade.

The present disclosure provides various methods and systems for treating an ultrasonic end effector or blade such as, for example, the end effector 12 to increase a resistance of the end effector or blade to crack or fracture propagation. In various instances, an ultrasonic end effector or blade such as, for example, the end effector 12 can be treated, or at least partially treated, to induce a residual compressive stress along at least one surface of the end effector or blade to improve the fatigue life of the end effector or blade once a crack or a fracture is formed.

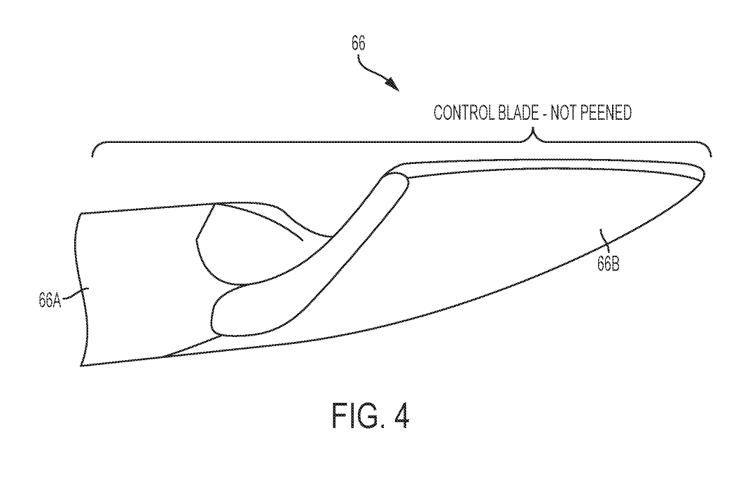

FIG. 4 shows an exemplary ultrasonic blade 66 prior to treatment. As noted above, ultrasonic blade 66 may comprise the end effector 12. As illustrated in FIG. 4, the blade 66 may include a balance region or support portion 66A and a treatment region or working end portion 66B that extends from the support portion 66A. Ultrasonic energy can be transmitted to the working end portion 66B through the support portion 66A.

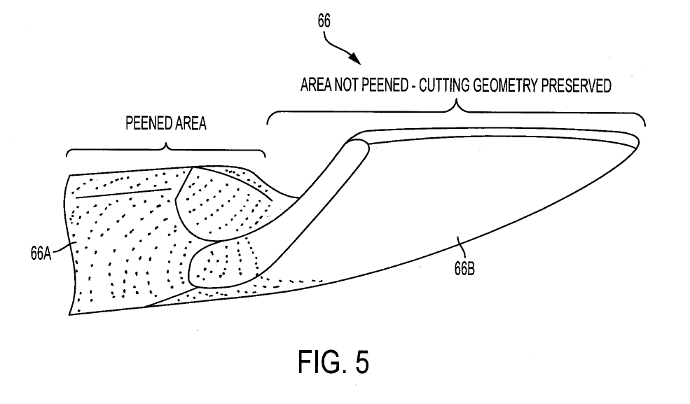

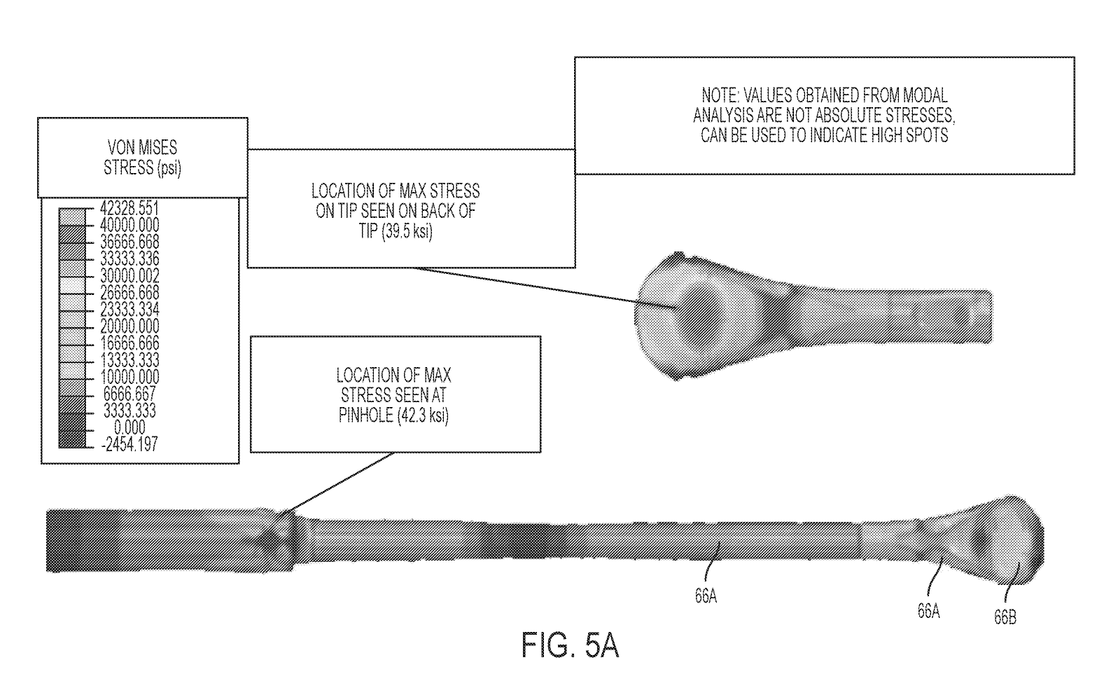

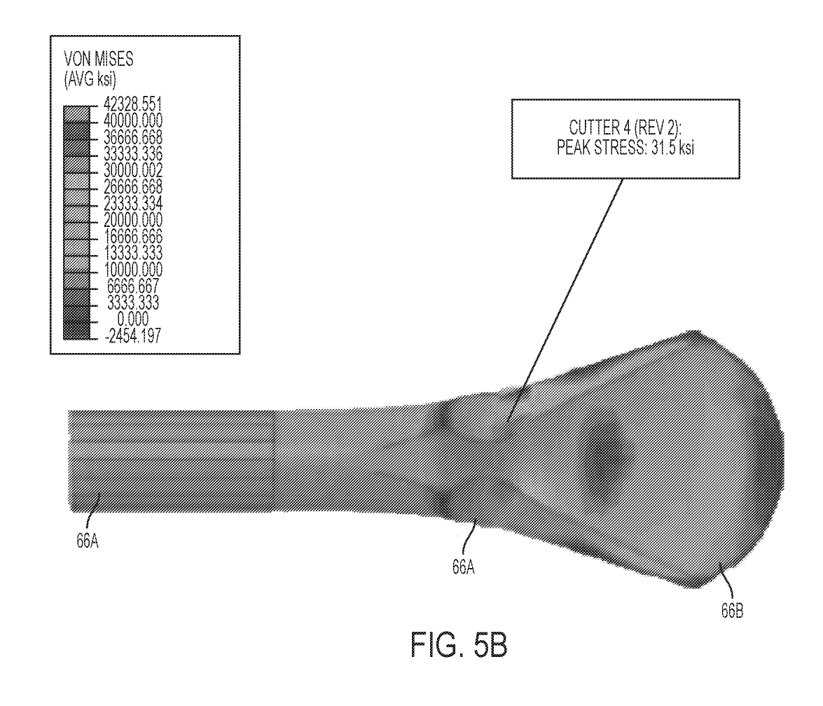

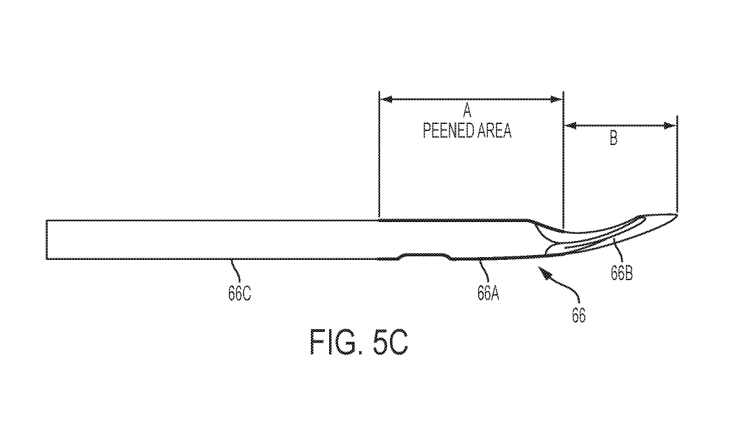

FIG. 5 shows the blade 66 after treatment. In at least one instance, as illustrated in FIG. 5, the support portion 66A can receive the treatment while the working end portion 66B of the blade 66 remains untreated. In some instances, depriving the working end portion 66B from the treatment can be desirable to sustain the mass symmetry of the working end portion 66B of the ultrasonic blade 66.

In various instances, a peening treatment can be selectively applied to one or more surfaces at one or more portions of the blade 66 to induce a residual compressive stress in such surfaces. In one instance, the peening treatment can be selectively applied to one or more surfaces at one or more portions of the blade 66 that are subject to relatively higher stresses than other portions of the blade 66 as the blade 66 resonates at ultrasonic frequencies. For example, as illustrated in FIG. 5A, the peening treatment can be applied to one or more surfaces of the support portion 66A.

FIGS. 3A-3C illustrate various embodiments wherein the peening treatment is applied to various sections of an ultrasonic transmission assembly. The ultrasonic transmission assemblies 11′, 11″, and 11′″ in FIGS. 3A, 3B, and 3C, respectively, are similar in many respects to the ultrasonic transmission assembly 11 in FIG. 3. Like elements are designated with like numbers, while the elements which differ slightly are designated by like-primed numbers. In one embodiment, as illustrated in FIG. 3A, a support portion or balance region 28′ of the ultrasonic transmission assembly 11′ receives a peening treatment while a treatment region 26 remains untreated. In this embodiment, the entire balance region 28′ is peened starting from a balance node 22 at a distal end of the waveguide 14 and ending at a distal end of the balance region 28′. Alternatively, in certain instances, only a portion of a balance region receives the peening treatment. For example, as illustrated in the embodiment of FIG. 3B, only a proximal portion of a balance region 28″ of ultrasonic transmission assembly 11″ is peened while the distal portion of the balance region 28″ remains untreated or unpeened. In the embodiments illustrated in FIGS. 3A and 3B, the treatment region 26 does not receive the peening treatment. In certain instances, a proximal portion of a treatment region 26 may receive the peening treatment while a distal portion of the treatment region 26 remains untreated or unpeened. The proximal portion of the treatment region 26 can be peened exclusively, with only a portion of the balance region 28, or with the entire balance region 28. For example, as illustrated in FIG. 3C, a proximal portion of treatment region 26′″ of the ultrasonic transmission assembly 11′″ is peened along with an entire balance region 28′″.

In at least one example, the peening treatment can be applied to one or more surfaces at a portion of the blade 66 that is subject to a high propensity of contact with hard objects while the blade 66 is vibrating at the ultrasonic frequencies. The hard objects can, for example, be surgical devices used in concert with the blade 66 in a surgical procedure. In at least one example, as depicted in FIG. 5B, the peening treatment is applied to one or more surfaces at a portion of the blade 66 that is subject to a combination of the high propensity of contact and the high stress such as, for example, the support portion 66A. In at least one example, the one or more surfaces that are subjected to the peening treatment are located at a portion of the blade 66 positioned between a distal unpeened working portion 66B and a proximal unpeened support portion 66C, as illustrated in FIG. 5C.

In one embodiment, the peened support portion 66A comprises a length greater than the distal unpeened working portion 66B. In one instance, as illustrated in FIG. 5C, the peened support portion 66A may comprise a length “A” and the unpeened working portion 66B may comprise a length “B”. In certain instances, the ratio of the length “A” to the length “B” can be selected from a range of about 0.6, for example, to about 0.95, for example. In certain instances, the ratio of the length “A” to the length “B” can be selected from a range of about 0.7, for example, to about 0.95, for example. In certain instances, the ratio of the length “A” to the length “B” can be selected from a range of about 0.8, for example, to about 0.95, for example. In alternative embodiments, the peened support portion 66A comprises a length less than the distal unpeened working portion 66B, for example.

In various instances, the peening and/or shot peening can be applied to one or more surfaces of the blade 66 such as, for example, a surface of the support portion 66A. In certain instances, the treated surface is plastically deformed. In certain instances, a treatment method of the present disclosure comprises producing a plurality of indentations along one or more surfaces of the support portion 66A.

In certain instances, a treatment method may comprise bombardment of a selected surface of the blade 66 with shot peening media. In certain instances, the shot peening media may be comprised of cast iron, cast steel, stainless steel, zirconia, or combinations thereof. In at least one example, the shot peening media may be comprised of a mixture of zirconia and stainless steel. In certain instances, the shot peening media may be comprised, or at least partially comprised, of a non-metallic material such as, for example, a ceramic material. In at least one example, the shot peening media may comprise a mixture of metallic media and non-metallic media.

In certain instances, the shot peening media may comprise one or more shapes. Examples of suitable shapes include spherical shapes, cylindrical shapes, diamond shapes, egg shapes, rod shapes, and/or cubic shapes. Shot peening media with shapes other than those described herein are contemplated by the present disclosure.

In at least one example, the shot peening media consists of particles of cast iron, cast steel, or stainless steel in various mesh sizes. In at least one example, the shot peening media comprise round particles. In at least one example, the shot peening media comprise rod-shaped particles. In one example, the shot peening media can be steel cut wires. In a preferred example, the shot peening media comprise ceramic beads. The ceramic beads may comprise smooth outer surfaces, which may reduce stress fracture initiation sites that may result from the increased surface roughness caused by the peening process, as discussed in greater detail below.

In certain instances, the bombardment of a selected blade surface with the shot peening media can be achieved by employing a high velocity stream of air to propel the shot peening media toward a selected blade surface. The velocity of the air stream and, in turn, the bombardment velocity of the peening media propelled by the air stream, may be adjusted to optimize the effects of the peening process on the selected blade surface. In certain instances, the high velocity stream of air can be generated by releasing pressurized air. The pressurized air may comprise any pressure selected from a range of about 10 psi to about 100 psi, for example. In at least one example, the pressurized air may comprise any pressure selected from a range of about 25 psi to about 50 psi, for example.

In certain instances, the shot peening media can be aimed at the blade surface at a trajectory that forms a predetermined bombardment angle with the target surface. The predetermined angle can be adjusted to optimize the effects of the peening process on the selected blade surface. In certain instances, the angle can be any angle selected from a range of about 50 degrees to about 150 degrees, for example. In at least one example, the angle can be any angle selected from a range of about 80 degrees to about 110 degrees.

The treatment or exposure time of the selected blade surface to the shot peening media can be adjusted to optimize the effects of the peening process on the selected blade surface. In certain instances, the exposure time can be any time selected from a range of about 0.1 second to about 10 seconds, for example. In at least one example, the exposure time can be any time selected from a range of about 1 second to about 6 seconds. In certain instances, the bombardment with the shot peening media can be performed in multiple stages. Each stage may employ one or more shot peening media. The shot peening media employed in various stages can differ in material composition, geometry, shape, and/or surface properties. The various stages may comprise the same, similar, or different exposure times, bombardment angles, and/or bombardment velocities.

In various instances, the above-described treatments may yield a fatigue resistant ultrasonic surgical blade that includes a layer of residual compressive stress along a surface of the blade. For example, as illustrated in FIG. 5, the resulting blade 66 may include a layer of residual compressive stress along a surface of the support portion 66A. The treatment increases the resistance of the treated surface of the support portion 66A to crack or fracture propagation thereby yielding an increase in the fatigue life of the blade 66. In various instances, as illustrated in FIG. 5, the blade 66 may include a peened support portion 66A and an unpeened working end portion 66B which extends from the peened support portion 66A, for example.

In certain instances, as illustrated in FIG. 5C, the layer of residual compressive stress in a treated blade surface may comprise a compressive stress depth selected from a range of about 0.001 inch to about 0.01 inch. In at least one example, the compressive stress depth can be any depth selected from a range of about 0.003 inch to about 0.006 inch. In at least one example, the compressive stress depth can be any depth selected from a range of about 0.004 inch to about 0.005 inch. In at least one example, the compressive stress depth can be 0.0045 inch.

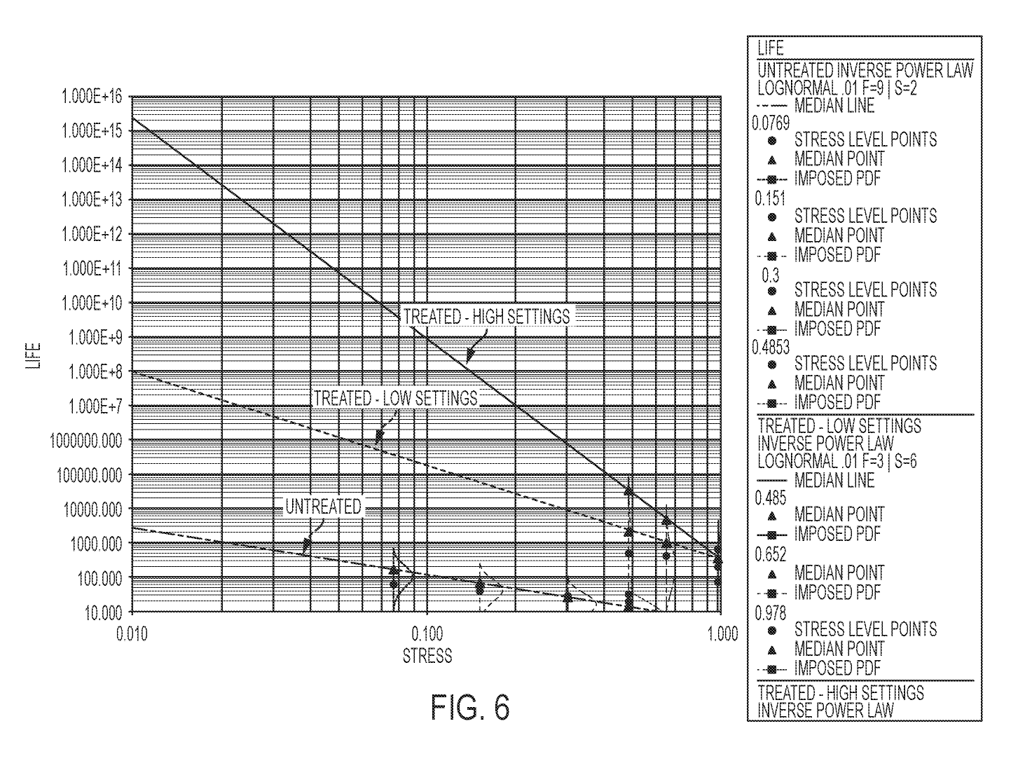

FIG. 6 illustrates the effects of the above-described treatment methods by comparing the ability of treated blades verses untreated blades to withstand breakage while vibrating at ultrasonic frequencies and maintaining contact with a metal object that is configured to apply a force against the vibrating blades. FIG. 6 shows a life stress plot of several treated and untreated blades. The time to breakage is shown on the Y-axis. The force applied by the metal object to the blades is shown on the X-axis. The three curves compare an untreated blade, a blade treated with low coverage or at a low treatment setting, and a blade treated with high coverage or a high treatment setting. The coverage level or treatment setting corresponds to the level of the treatment. In at least one example, the coverage level or the treatment setting corresponds to the time the blade is subjected to the treatment. In the embodiment illustrated in FIG. 6, the treatment method employed to treat the blades was shot peening. As clearly indicated by the life stress plot of FIG. 6, the above-described treatment methods increase the ability of the treated blades to withstand breakage.

Notably, higher peening intensity and longer peening time may increase the compressive stress depth in a treated blade surface thereby increasing the ability of the treated surface to resist stress fracture propagation However, peening may increase the roughness of a treated blade surface; and a roughened surface may have a negative effect on stress cracking. The negative effect is caused by an increase stress fracture initiation sites. The degree of surface roughness is proportional to the peening intensity and duration. Accordingly, the parameters of the peening process need to be carefully selected, as described above, to increase the ability of the treated surface to resist stress fracture propagation while maintaining a tolerable surface roughness.

In one embodiment, an optimum fatigue life of a peened blade has been achieved when a selected surface of the blade has been treated by bombarding the selected blade surface with shot peening media comprising zirconia and stainless steel for a duration selected from a range of about 1 second to about 6 seconds. The shot peening media was propelled towards the selected blade surface at an angle selected from a range of about 80 degrees to about 110 degrees via a high velocity stream of air generated by releasing pressurized air comprising a pressure selected from a range of about 25 psi to about 50 psi. The coverage level was about 200%. The selected blade surface was placed at the center of the enclosure at a distance selected from a range of about 6 inches to about 12 inches from the shot peening media.

In certain instances, the shot peening media comprises a Conditioned Stainless Steel Cut Wre Shot AMS 2431/4C. The reader will appreciate that the shot peening media Conditioned Stainless Steel Cut Wire Shot with various sizes, shapes, and geometries.

In various instances, as described above, the coverage level can be 200%. In other words, the treatment can be repeated twice for a selected surface. In certain instances, the coverage level can be 300%, wherein the treatment is repeated three times for a selected surface.

In various instances, the selected blade surface can be placed at the center of the enclosure at a distance selected from a range of about 7 inches to about 11 inches from the shot peening media. In various instances, the selected blade surface can be placed at the center of the enclosure at a distance selected from a range of about 8 inches to about 10 inches from the shot peening media. In at least one example, the selected blade surface can be placed at the center of the enclosure at a distance of about 9 inches from the shot peening media.

As described above, the treatment time can be selected from a range of about 1 second to about 6 seconds. In certain instances, the treatment time can be selected from a range of about 2 seconds to about 5 seconds. In certain instances, the treatment time can be selected from a range of about 3 seconds to about 4 seconds. In certain instances, the treatment time can be 3.5 seconds. In certain instances, the treatment can be performed in sessions. Each session may comprise a treatment time selected from a range of about 1 second to about 6 seconds, for example.

As described above, the shot peening media was propelled towards the selected blade surface at an angle selected from a range of about 80 degrees to about 110 degrees. In certain instances, the angle can be any angle selected from a range of about 90 degrees to about 100 degrees. In certain instances, the angle can be about 95 degrees. In certain instances, the angle can be varied during the treatment. In at least one example, a first treatment session may comprise a first angle and a second treatment session may comprise a second angle different from the first angle. In at least one example, the shot peening media can be simultaneously propelled toward a selected surface of the blade at multiple angles.

As described above, a high velocity stream of air can be used to propel the peening shot media. In the embodiment described above, the high velocity stream of air is generated by releasing pressurized air comprising a pressure selected from a range of about 25 psi to about 50 psi. In certain instances, the pressure is selected from a range of about 30 psi to about 40 psi. In certain instances, the pressure is about 35 psi, for example. In various instances, a different fluid can be employed to propel the shot peening media toward a selected blade surface.

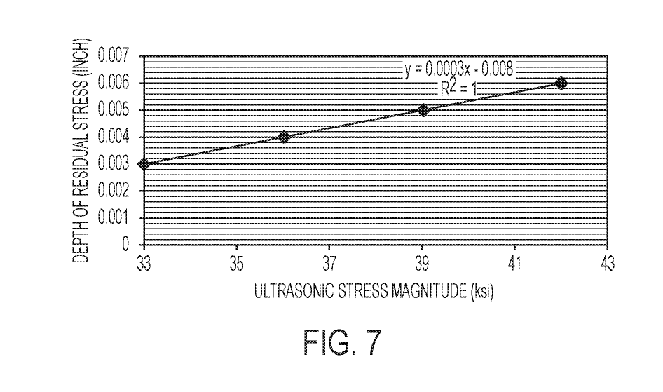

FIG. 7 is a plot showing a correlation between the depth of the residual compressive stress induced in an ultrasonic blade and the robustness of the treated blade. The depth of the residual compressive stress in inches is plotted on the Y-axis. The robustness of the treated blade, represented by an ultrasonic stress magnitude in kilopound per square inch (Ksi), is plotted on the X-axis. The residual compressive stress follows the transfer function:

Depth(compressive stress,inch)=a+m*(ultrasonic stress magnitude,Ksi)

In certain instances, “a” is any value selected from a range of about −0.0006 to about −0.010 and “m” is any value selected from a range of about 0.00028 to about 0.00038. In one example, “a” is any value selected from a range of about −0.0007 to about −0.0009. In one example, “a” is about −0.0008. In certain instances, “m” is about 0.00035.

As described above, the treatment methods of the present disclosure may increase the roughness of a treated blade surface, which may have a negative effect on stress cracking. To counter such negative effects, an additional step can be added to the treatment methods described above to smooth the treated surface. A tumbling process can be employed to smooth the treated blade surface. In one example, fiber glass is employed as a smoothing medium. Other smoothing media are contemplated by the present disclosure. Alternatively, a polishing process can employed to smooth the treated blade surface, for example.

Various other peening techniques can be employed to treat the support portion 66A of the blade 66 such as, for example, laser shock peening. U.S. Patent Application Publication No. 2009/0043228, entitled LASER SHOCK PEENING OF MEDICAL DEVICES, and filed Aug. 6, 2007, illustrates various suitable laser shock peening techniques. The entire disclosure of U.S. Patent Application Publication No. 2009/0043228 is hereby incorporated by reference herein.

### EXAMPLES

### Example 1

A method of treating an ultrasonic surgical blade to improve resistance of the ultrasonic surgical blade to fracture propagation, comprising providing an ultrasonic blade and inducing a residual compressive stress along at least one surface of the ultrasonic blade.

### Example 2

The method of Example 1, wherein the residual compressive stress is proportional to an ultrasonic stress magnitude.

### Example 3

The method of Examples 1 or 2, wherein the residual compressive stress follows the transfer function: Depth (compressive stress, inch)=a+m*(ultrasonic stress magnitude, Ksi), where in “a” is in the range of about −0.0006 to about −0.010 and “m” is in the range of about 0.00028 to about 0.00038.

### Example 4

The method of Examples 1 or 2 or 3, wherein the residual compressive stress comprises a compressive stress depth that ranges from about 0.006 inch to about 0.010 inch.

### Example 5

The method of Examples 1 or 2 or 3 or 4, wherein the residual compressive stress is imparted to selective areas.

### Example 6

The method of Examples 1 or 2 or 3 or 4 or 5, comprising bombarding the at least one surface with shot peening media comprising zirconia and stainless steel at a distance in a range of about 6 inches to about 12 inches for an exposure time in a range of about 1 second to about 6 seconds by propelling the shot peening media toward the at least one surface at an angle of attack in a range of about 80 degrees to about 110 degrees via a high velocity stream of air generated by releasing air pressurized in a range of about 25 psi to about 50 psi, wherein the at least one surface is bombarded twice for a coverage level of 200 percent.

### Example 7

A method of treating an ultrasonic surgical blade to improve resistance of the ultrasonic surgical blade to fracture propagation, comprising providing an ultrasonic blade, treating at least one surface of the ultrasonic blade by inducing a residual compressive stress along at least one surface of the ultrasonic blade, and smoothing the treated surface.

### Example 8

The method of Example 7, wherein a fiber glass is used as a smoothing medium to smooth the treated surface.

### Example 9

The method of Examples 7 or 8, wherein the smoothing is done through tumbling.

### Example 10

The method of Examples 7 or 8, wherein the smoothing is done by polishing

### Example 11

A method of treating an ultrasonic surgical blade to improve resistance of the ultrasonic surgical blade to fracture propagation, comprising providing an ultrasonic blade, and inducing a residual compressive stress along at least one surface of the ultrasonic blade.

### Example 12

The method of Example 11, wherein the ultrasonic blade comprises a working end portion, and wherein the at least one surface is outside the working end portion.

### Example 13

The method of Examples 11 or 12, wherein inducing the residual compressive stress comprises producing a plurality of indentations on the at least one surface.

### Example 14

The method of Examples 11 or 12 or 13, wherein inducing the residual compressive stress comprises peening the at least one surface.

### Example 15

The method of Examples 11 or 12 or 13 or 14, wherein inducing the residual compressive stress comprises shot peening the at least one surface.

### Example 16

The method of Example 15, wherein the shot peening comprises bombarding the at least one surface with a plurality of spherical shots.

### Example 17

The method of Example 16, wherein the spherical shots comprise rounded particles.

### Example 18

The method of Examples 11 or 12 or 13 or 14 or 15, wherein inducing the residual compressive stress comprises plastically deforming the at least one surface.

### Example 19

A fatigue resistant ultrasonic surgical blade prepared by a process, comprising providing an ultrasonic blade, and inducing a residual compressive stress along at least one surface of the ultrasonic blade.

### Example 20

The fatigue resistant ultrasonic surgical blade of Example 19, wherein the ultrasonic blade comprises a working end portion, and wherein the at least one surface is outside the working end portion.

### Example 21

The fatigue resistant ultrasonic surgical blade of Examples 19 or 20, wherein inducing the residual compressive stress comprises producing a plurality of indentations on the at least one surface.

### Example 22

The fatigue resistant ultrasonic surgical blade of Examples 19 or 20 or 21, wherein inducing the residual compressive stress comprises peening the at least one surface.

### Example 23

The fatigue resistant ultrasonic surgical blade of Examples 19 or 20 or 21 or 22, wherein inducing the residual compressive stress comprises shot peening the at least one surface.

### Example 24

The fatigue resistant ultrasonic surgical blade of Example 23, wherein the shot peening comprises bombarding the at least one surface with a plurality of spherical shots.

### Example 25

The fatigue resistant ultrasonic surgical blade of Example 24, wherein the spherical shots comprise rounded particles.

### Example 26

The fatigue resistant ultrasonic surgical blade of Examples 19 or 20 or 21 or 22 or 23, wherein inducing the residual compressive stress comprises plastically deforming the at least one surface.

### Example 27

An ultrasonic surgical instrument for use in a surgical procedure, the ultrasonic surgical instrument comprising a transducer, an ultrasonic surgical blade configured to vibrate at ultrasonic frequencies to treat tissue, wherein the ultrasonic surgical blade includes a peened support portion comprising a layer of residual compressive stress along at least one surface of the peened support portion and an unpeened working end portion extending from the peened support portion.

### Example 28

The ultrasonic surgical instrument of Example 27, wherein the at least one surface is disposed at a region of the support portion that is subject to highest stress while the ultrasonic surgical blade vibrates at the ultrasonic frequencies.

### Example 29

An ultrasonic surgical instrument for use in a surgical procedure, the ultrasonic surgical instrument comprising an ultrasonic transmission waveguide operably coupleable to a transducer and an ultrasonic generator, and an ultrasonic end effector operably coupled to the ultrasonic transmission waveguide, wherein the ultrasonic end effector comprises a support portion and a working portion extending from the support portion to treat tissue, wherein at least one surface of at least one of the support portion or the working portion comprises a peened layer of residual compressive stress to increase the resistance of the ultrasonic end effector to fracture propagation and to increase the fatigue life of the ultrasonic end effector.

### Example 30

The ultrasonic surgical instrument of Example 29, wherein the at least one surface of the at least one of the support portion or the working portion comprises one or more surfaces having a high propensity of contacting hard objects when the ultrasonic end effector is vibrating at ultrasonic frequencies.

Although the various embodiments of the devices have been described herein in connection with certain disclosed embodiments, many modifications and variations to those embodiments may be implemented. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

While this invention has been described as having exemplary designs, the present invention may be further modified within the spirit and scope of the disclosure. This application is therefore intended to cover any variations, uses, or adaptations of the invention using its general principles.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A method of treating an ultrasonic surgical blade to improve resistance of the ultrasonic surgical blade to fracture propagation, the method comprising:
providing an ultrasonic blade; and
inducing a residual compressive stress along at least one surface of the ultrasonic blade.

2. The method of claim 1, wherein the ultrasonic blade comprises a working end portion, and wherein the at least one surface is outside the working end portion.

3. The method of claim 1, wherein the step of inducing the residual compressive stress comprises producing a plurality of indentations on the at least one surface.

4. The method of claim 1, wherein the step of inducing the residual compressive stress comprises peening the at least one surface.

5. The method of claim 1, wherein the step of inducing the residual compressive stress comprises shot peening the at least one surface.

6. The method of claim 5, wherein the shot peening comprises bombarding the at least one surface with a plurality of spherical shots.

7. The method of claim 6, wherein the spherical shots comprise rounded particles.

8. The method of claim 1, wherein the step of inducing the residual compressive stress comprises plastically deforming the at least one surface.

9. A fatigue resistant ultrasonic surgical blade prepared by a process comprising the steps of:
providing an ultrasonic blade; and
inducing a residual compressive stress along at least one surface of the ultrasonic blade.

10. The fatigue resistant ultrasonic surgical blade of claim 9, wherein the ultrasonic blade comprises a working end portion, and wherein the at least one surface is outside the working end portion.

11. The fatigue resistant ultrasonic surgical blade of claim 9, wherein the step of inducing the residual compressive stress comprises producing a plurality of indentations on the at least one surface.

12. The fatigue resistant ultrasonic surgical blade of claim 9, wherein the step of inducing the residual compressive stress comprises peening the at least one surface.

13. The fatigue resistant ultrasonic surgical blade of claim 9, wherein the step of inducing the residual compressive stress comprises shot peening the at least one surface.

14. The fatigue resistant ultrasonic surgical blade of claim 13, wherein the shot peening comprises bombarding the at least one surface with a plurality of spherical shots.

15. The fatigue resistant ultrasonic surgical blade of claim 14, wherein the spherical shots comprise rounded particles.

16. The fatigue resistant ultrasonic surgical blade of claim 9, wherein the step of inducing the residual compressive stress comprises plastically deforming the at least one surface.

17. An ultrasonic surgical instrument for use in a surgical procedure, the ultrasonic surgical instrument comprising:
a transducer;
an ultrasonic surgical blade configured to vibrate at ultrasonic frequencies to treat tissue, the ultrasonic surgical blade comprising:
a peened support portion comprising a layer of residual compressive stress along at least one surface of the peened support portion; and
an unpeened working end portion extending from the peened support portion.

18. The ultrasonic surgical instrument of claim 17, wherein the at least one surface is disposed at a region of the support portion that is subject to highest stress while the ultrasonic surgical blade vibrates at the ultrasonic frequencies.

19. An ultrasonic surgical instrument for use in a surgical procedure, the ultrasonic surgical instrument comprising:
an ultrasonic transmission waveguide operably coupleable to a transducer and an ultrasonic generator; and
an ultrasonic end effector operably coupled to the ultrasonic transmission waveguide, wherein the ultrasonic end effector comprises:
a support portion; and
a working portion extending from the support portion to treat tissue; and
wherein at least one surface of at least one of the support portion or the working portion comprises a peened layer of residual compressive stress to increase the resistance of the ultrasonic end effector to fracture propagation and to increase the fatigue life of the ultrasonic end effector.

20. The ultrasonic surgical instrument of claim 19, wherein the at least one surface of the at least one of the support portion or the working portion comprises one or more surfaces having a high propensity of contacting hard objects when the ultrasonic end effector is vibrating at ultrasonic frequencies.
